(19) United States
(12) Patent Application Publication
(10) Pub. No.: US 2010/0042199 A1
(43) Pub. Date: Feb. 18, 2010

(54) DILATION BALLOON CATHETER AND METHODS OF USE THEREOF

(76) Inventor: David G. Burton, Bloomington, IN (US)

Correspondence Address: BRINKS HOFER GILSON & LIONE/CHICAGO/COOK, PO BOX 10395, CHICAGO, IL, 60610, US

(21) Appl. No.: 12/508,976

(22) Filed: Jul. 24, 2009

Related U.S. Application Data
(60) Provisional application No. 61/089,764, filed on Aug. 18, 2008.

(51) Int. Cl. A61F 2/84; A61M 29/02

(52) U.S. Cl. 623/1.11; 604/103.06

(57) ABSTRACT

The present invention relates to medical devices for dilating or enlarging strictures or narrowed regions of body vessels. Specifically, the present invention relates to a high pressure dilation balloon catheter that includes an elongate shaft extending between a proximal end and a distal end, the proximal end being adapted for attachment to a source of inflation fluid, and a first lumen extending through the shaft adapted for the passage of the inflation fluid; and a balloon disposed on the distal end of the shaft and having a balloon body extending between a proximal end and a distal end of the balloon. The balloon body includes an inner balloon layer, an outer balloon layer, a middle layer disposed between the inner balloon layer and the outer balloon layer and configured to be substantially free from adhesion to at least one of the inner balloon layer and the outer balloon layer, and a balloon chamber within the first layer, the balloon chamber being in a communication with the lumen of the shaft for inflating and deflating the balloon.

### RELATED APPLICATIONS

The present patent document claims the benefit of the filing date under 35 U.S.C. §119(e) of Provisional U.S. Patent Application Ser. No. 61/089,764, filed Aug. 18, 2008, which is hereby incorporated by reference.

### BACKGROUND

A variety of body lumens are subject to undesired strictures or narrow regions. For example, blood vessels can be blocked or narrowed by atherosclerosis, while esophageal strictures can arise from individual anatomical differences, or from diseases such as connective tissue disorder.

Procedures for dilating or enlarging such strictures or narrowed regions often entail the use of a balloon dilation catheter. In general, such catheters include a deflated balloon which can be positioned across a particular stricture or narrowed region, and which is then inflated with an inflation fluid in order to widen the lumen without trauma to the wall of the lumen.

Conventional dilation balloons fall into high, medium, and low pressure ranges. Low pressure balloons are those that have burst pressures below 6 atmospheres (ATM) (6.1×10⁵ Pascals). Medium pressure balloons are those that have burst pressures between 6 and 15 ATM (6.1×10⁵ and 1.2×10⁶ Pa). High pressure balloons are those that have burst pressures above 15 ATM (1.2×10⁶ Pa) and as high as 30 ATM. The term “burst pressure” refers to the maximum pressure which can be slowly applied to the balloon (at a specific temperature and for a specified amount of time (e.g., seconds or minutes)) without causing it to rupture or burst. Burst pressure is determined by such factors as the wall thickness and tensile strength of the balloon material.

High pressure balloons are desirable because they have the ability to exert more force and “crack” hard lesions. High pressure balloons are useful in high pressure procedures, such as Percutaneous Transluminal Angioplasty (PTA) in the peripheral vasculature, including the iliac, femoral, ilio-femoral, popliteal and renal arteries, and for the treatment of obstructive lesions of native or synthetic arteriovenous dialysis fistulae. High pressure balloons are also useful in stent deployment.

A biocompatible metal stents are often used to prop open blocked coronary arteries, and keeping them from re-closing after balloon angioplasty. In an exemplary procedure, a balloon of appropriate size and pressure is first used to open the lesion. The process is then repeated with a stent crimped onto a high pressure balloon. The stent is deployed when the balloon is inflated. A medium to high pressure balloon is preferable for stent deployment because the stent must be forced against the artery's interior wall so that it will fully expand, thereby precluding the ends of the stent from projecting into the arterial channel, which may inhibit flow there through and encourage the formation of thrombus.

High pressure balloon materials are typically stiffer than conventional medium or low pressure balloon materials. Whereas medium or low pressure balloons use materials such as polyethylene, high pressure balloons use materials such as Nylon 12 or PET. See, for example, U.S. Pat. No. 4,490,421, U.S. Pat. No. Re. 32,983, U.S. Pat. No. Re. 33,561, and EP 0135990, which disclose a high molecular weight, biaxially oriented, flexible, polymeric balloon with a tensile strength of at least 31,714 psi (218.86 MPa), which can be made of PET, which are incorporated by reference herein in their entirety. See, also, U.S. Pat. No. 5,264,260, which discloses a PET balloon, optionally melt blended or mixed with other polymeric or nonpolymeric materials, having an intrinsic viscosity of less than or equal to 0.6 dl/g and a calculated radial tensile strength greater than about 25,000 psi (172 MPa) and is also incorporated by reference herein in its entirety.

In general, improvements have been made to conventional high pressure balloons over the years. However, because these balloons are subject to the application of high pressure, these balloons are still prone to puncture or tearing, such as circular tearing of the balloons under burst pressure. Moreover, when these balloons burst in a constricted state, they often tear along a circumferential path that may lead to separation of the balloon into two or more pieces. As a consequence, forceps or other device may need to be inserted into a patient to remove the balloon pieces, thus, requiring more complicated and/or longer procedures.

As such, there still exists a need in the industry for high pressure balloons which display improved puncture and tearing resistance, when compared to the conventional high pressure balloons, while maintaining sufficient burst strength.

### SUMMARY

In one embodiment, the invention relates to a dilation balloon catheter. The dilation balloon catheter includes an elongate shaft extending between a proximal end and a distal end, the proximal end being adapted for attachment to a source of inflation fluid, and a lumen extending through the shaft adapted for the passage of the inflation fluid; and a balloon disposed on the distal end of the shaft and having a balloon body extending between a proximal end and a distal end of the balloon. The balloon body includes: an inner balloon layer; an outer balloon layer, a middle layer disposed between the inner balloon layer and the outer balloon layer and configured to be substantially free from adhesion to at least one of the inner balloon layer and the outer balloon layer, and a balloon chamber within the inner balloon layer, the balloon chamber being in a communication with the lumen of the shaft for inflating and deflating the balloon. The inner balloon layer and the outer balloon layer may each be formed from a substantially non-compliant and non-porous elastomeric material, such as Nylon (Nylon 12), polyether block amide (PEBAX), PEBAX 4033, PEBAX 5533, PEBAX 6333, and poly(ethylene terephthalate) (PET).

The balloon may have a predetermined inflated diameter in the range of from about 2 millimeters to about 30 millimeters. The inner balloon layer may be configured to have an inflated diameter smaller than the inflated diameter of the outer balloon layer. The inner balloon layer may have a thickness from about 0.014 millimeters to about 0.060 millimeters. The outer balloon layer may have a thickness from about 0.008 millimeters to about 0.047 millimeters. The middle layer may have thickness of about 0.010 millimeters to about 0.070 millimeters. The proximal and the distal ends of the balloon body may each be tapered.

In certain embodiments, the inner and the outer balloon layers may comprise different materials. The inner and the outer balloon layers may also comprise different thicknesses. The balloon may be configured to exert an outward pressure of from about 12 atmospheres to about 30 atmospheres when inflated.

The shaft of the dilation balloon catheter may further include a wireguide lumen extending through at least a portion thereof. The wireguide lumen may be disposed adjacent to the inflation lumen of the shaft. The wireguide lumen may extend through a substantial portion of the shaft and terminate in a proximal port near the proximal end of the shaft. The shaft may include a port through a side wall thereof in communication with the wireguide lumen, the port being located proximal of the balloon and a substantial distance from the proximal end of the shaft. The shaft may include either one or both of these proximal ports. The wireguide lumen may include a wire guide coaxially and movably disposed there through.

In another embodiment, the invention relates to a method for dilating a vessel stricture by providing a dilation balloon catheter as described above; positioning the balloon within or near the vessel stricture; and inflating the balloon to dilate or widen the vessel stricture.

In yet another embodiment, the method may further include the steps of providing a stent; compressing the stent about the balloon when the balloon is in an uninflated state; and expanding the balloon to expand and deploy the stent.

In yet another embodiment, the invention relates to a dilation balloon catheter. The dilation balloon catheter includes an elongate shaft extending between a proximal end and a distal end, the proximal end being adapted for attachment to a source of inflation fluid, and a lumen extending through the shaft adapted for the passage of the inflation fluid; a balloon disposed on the distal end of the shaft and having a balloon body extending between a proximal end and a distal end of the balloon. The balloon body includes an inner balloon layer, an outer balloon layer, at least one fluoropolymer layer disposed between the inner balloon layer and the outer balloon layer, and a balloon chamber within the inner balloon layer, the balloon chamber being in a communication with the lumen of the shaft for inflating and deflating the balloon. The fluoropolymer may be extended polytetrafluorethylene.

### BRIEF DESCRIPTION OF THE DRAWINGS

The devices, systems and methods may be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIGS. 1A-1C depict an exemplary dilation balloon catheter device;

FIG. 2 depicts dilation balloon portion of the device of FIGS. 1A-1C;

FIG. 3 shows a cross-sectional view though E-E of the exemplary dilation balloon of FIG. 2;

FIGS. 4A-4C depicts a coaxial configuration of the shaft of an exemplary dilation balloon catheter device;

FIG. 5 depicts an exemplary inflation device; and

FIG. 6 depicts a standard single lumen balloon tubing;

FIG. 7 depicts an exemplary dilation balloon catheter device deployed in a body lumen; and

FIG. 8 depicts yet another embodiment of the exemplary dilation balloon catheter deployed in a body lumen.

### DETAILED DESCRIPTION OF THE DRAWINGS AND THE PRESENTLY PREFERRED EMBODIMENTS

The present invention relates to medical devices, and more specifically to dilation catheter devices, which can be used for dilation (i.e., mechanical widening) of strictures, during high pressure procedures, such as Percutaneous Transluminal Angioplasty (PTA) in the peripheral vasculature, including the iliac, femoral, ilio-femoral, popliteal and renal arteries, and for the treatment of obstructive lesions of native or synthetic arteriovenous dialysis fistulae. The devices of the present invention can also be used for post-dilation of balloon expandable and self-expanding stents in the peripheral vasculature and other bodily lumens of a patient.

Embodiments of the dilation catheter devices described herein generally include a shaft adapted for the passage of the inflation fluid there through and a balloon disposed on the distal end of the shaft. The balloon has a balloon body that includes two separate balloon layers (inner balloon layer and outer balloon layer) and a middle layer disposed between the inner balloon layer and the outer balloon layer and configured to be substantially free from adhesion to at least one of the inner balloon layer and the outer balloon layer. The middle layer may comprise, for examples a fluoropolymer, such as ePTFE.

It is believed that the inclusion of a middle layer, which is configured to be substantially free from adhesion to at least one of the inner balloon layer and the outer balloon layer and which may comprise ePTFE, between the inner and the outer balloons layers of the dilation balloon catheter device provides a balloon catheter suitable for use in high pressure applications. This is because the middle layer comprising, for example, ePTFE advantageously allows the two balloon layers to expand independently of each other during inflation of the balloon, while maintaining contact with each other. The independent movement may occur because the middle layer prevents adhesion of the inner and the outer balloon layers. Because all the layers (i.e., inner balloon layer, the middle layer, and the outer balloon layer) are in contact with each other during inflation but moving independently, the overall burst will be significantly higher than a single layer balloon having dimensions (e.g., thickness) equivalent to the dimensions of the two balloon layers combined. Specifically, the highest stresses during pressurization occur at the inside diameter of the balloon. Because each balloon layer moves independently of the other, there are essentially two inside diameters of the two balloon layers (i.e., much larger surface area) to distribute the stresses. Also, it is believed that some localized areas of increased stress or weakness in the material in the inner balloon layer may get distributed over a larger area of the outer balloon layer.

In addition, the middle layer can function to resist circular tearing of the balloon under burst pressure.

Definitions:

Unless otherwise indicated, all ordinary words and terms used herein shall take their customary meaning as defined in The New Shorter Oxford English Dictionary, 1993 edition. All technical terms shall take on their customary meaning as established by the appropriate technical discipline utilized by those normally skilled in that particular art area. All medical terms shall take their meaning as defined by Stedman's Medical Dictionary, 27^{th} edition.

The terms “about” or “substantially” used with reference to a quantity includes variations in the recited quantity that are equivalent to the quantity recited, such as an amount that is insubstantially different from a recited quantity for an intended purpose or function.

The terms “adapted for” or “configured to” while referring to an element of the dilation balloon catheter described herein mean that the element is changed, modified, or specifically designed so that it is suitable to perform a specified or desired function.

As used herein, “disposed” means placed or arranged in a particular order to define the relationship between elements or components of a device. The term “disposed” can include, without being limited to, terms, such as, placed, arranged, distributed, or incorporated.

The term “proximal” refers to an area nearer to a point of reference such as an origin or a point of attachment. In this application the term proximal refers to an area nearer to the physician.

The term “distal” refers to an area further from a point of reference, e.g., further from a physician.

The term “non-compliant” refers to a type of material that is used to form the balloon portion of the balloon catheter described herein. “Non-compliant” material may be characterized by high stiffness, rigidity, and low compliance. The term “non-compliant,” throughout the instant specification, also refers to materials, which are substantially non-compliant (i.e., semi-compliant) or substantially non-elastic. These terms may be used interchangeably.

The term “shaft” refers to a tubular structure, such as, for example, a catheter.

The term “tubular” refers to the general shape of a device or an element of the device, which allows the device to carry fluid along a distance or fit within a tubular structure such as an artery.

The term “stent” refers to any device or structure that adds rigidity, expansion force or support to a tubular structure, such as vessel wall.

The term “stent graft” refers to a type of endoluminal prosthesis made of a tubular graft material and supported by at least one stent.

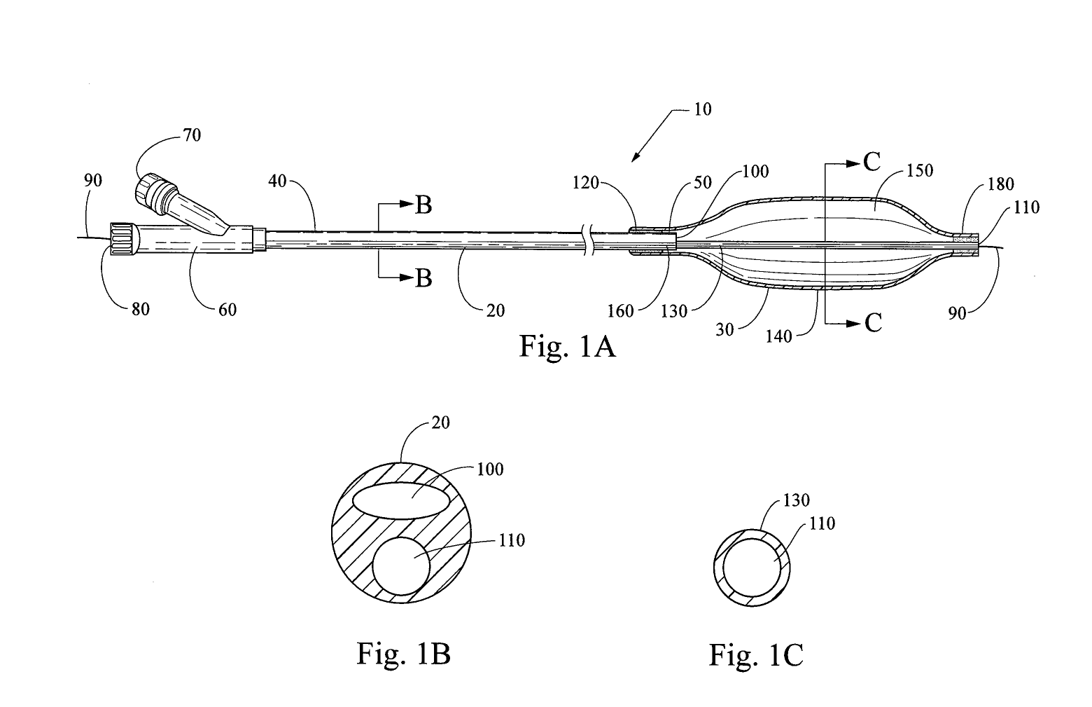

Referring to FIGS. 1A-1C, an exemplary embodiment of the present invention is shown and illustrates a high pressure dilation balloon catheter 10, which includes an elongate shaft 20 and a balloon 30 disposed on a distal end 50 of the shaft 20. As shown in the drawings, the balloon 30 comprises, in its fully inflated profile shape, a cylindrical working portion with an inflated diameter located between a pair of conical end portions, and proximal and distal legs (i.e., neck portions) extending from the conical portions and affixed to the shaft. The balloon in its deflated profile shape may have several pleats (not shown) that allow the balloon to be wrapped around the shaft to reduce its profile so as to facilitate advancement of the balloon catheter into the patient.

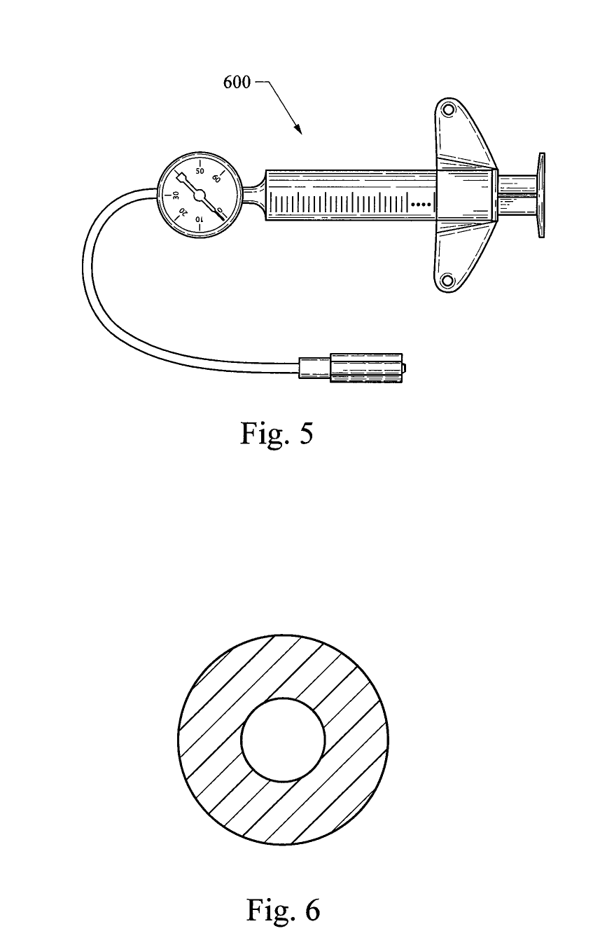

Specifically, the balloon catheter 10 includes an elongate shaft (i.e., tube) 20, which can be made from a flexible catheter tubing, such as Nylon. The shaft 20 is preferably tubular and extends between a proximal end 40 and a distal end 50, where the proximal end 40 can attach to a hub 60, which can include an inflation port 70, which then connects to a source of inflation fluid, i.e., inflation device (not shown). An exemplary inflation device, such as a syringe 600, is illustrated in FIG. 5.

As shown in FIG. 1A, the balloon catheter may be also adapted for use with optional ancillary instrumentation, such as a wire guide 90, where the hub 60 also includes a wireguide port 80 in communication with the wireguide lumen 110.

In a first illustrative embodiment, the wire-guided dilation balloon catheter 10 includes a shaft 20 that comprises a dual lumen shaft, best seen in FIG. 1B, which is a cross-section though B-B in FIG. 1A. In particular, the shaft 20 includes an inflation lumen 100 for the passage of the inflation fluid, and a wireguide lumen 110 to accommodate wire guide 90 that may be used in a procedure. The inflation lumen 100 terminates at the location near the proximal balloon bond 120 and is in fluid communication with the interior of the balloon for the delivery of the inflation fluid into the balloon 30. A single lumen shaft 130 extends from the main shaft 20 and through the balloon body 140 (FIGS. 1A and 1C) and is in communication with the wireguide lumen 110 of the shaft 20. FIG. 1C is a cross-section taken along line C-C of FIG. 1A. The shaft 130 terminates near the distal end 180 of the balloon 30 and can include a passageway via which the wire guide 90 may enter and exit the balloon catheter 10 to aid in cannulation or perform some other function. The inflation fluid, such as water or saline, for inflation of the balloon 30 is supplied via the main shaft 20 through the inflation lumen 100 and into the balloon chamber 150. The single lumen shaft 130 may be heat bonded to the distal end 160 of the shaft 20 or may be formed as a unitary structure.

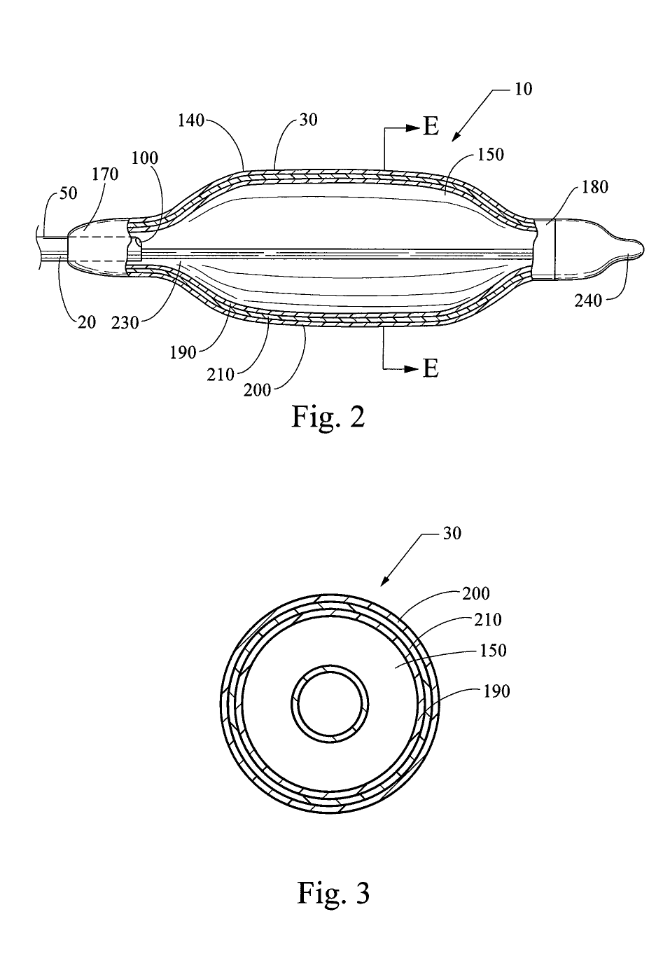

As mentioned above, the dilation balloon catheter 10 of this invention includes a balloon 30 disposed on a distal end 50 of the shaft 20. The illustrative balloon 30 of the balloon catheter 10 is shown in greater detail in FIGS. 2 and 3. The balloon 30 has a balloon body 140 extending between a proximal end 170 and a distal end 180 of the balloon 30. The balloon body 140 includes an inner balloon layer 190 and an outer balloon layer 200. The balloon body 140 also includes a middle layer 210 configured to be substantially free from adhesion to at least one of the inner balloon layer and the outer balloon layer, the middle layer comprising at least one layer of fluoropolymer, such as ePTFE 210 disposed between the inner and the outer balloon layers 190, 200, and a balloon chamber 150 within the inner balloon layer 190. The balloon chamber 150 remains in communication with the inflation lumen 100 of the shaft 20 for inflating and deflating the balloon 30. As illustrated in FIG. 3, which is a cross-section though E-E of FIG. 2, the middle layer 210 is disposed between the inner balloon layer 190 and the outer balloon layer 200.

Each of the balloon layers 190, 200 making up the balloon 30 can be formed to have a specific inflated diameter. Preferably, the inflated diameter of the inner balloon layer is slightly smaller than the inflated diameter of the outer balloon layer. The inner balloon's outer diameter is preferably smaller than the outer diameter of the outer balloon layer by approximately the sum of the wall thicknesses of the outer balloon and the middle layer (e.g., ePTFE layer).

The balloon portion 30 of the dilation balloon catheter 10, including the two balloon layers, can be formed of a balloon material that is preferably substantially inelastic, and stretches a relatively small amount under pressures of 15 atmospheres or more. Various materials may be used, including Nylon (e.g., Nylon 12), polymeric materials such as poly(ethylene terephthalate) (PET), PEEK, PEBAX material, or a block copolymer thereof. Other suitable materials may also be used.

The balloon 30 can be attached to the shaft 20 by variety of methods, including by inserting the distal end 50 of the shaft 20 into the proximal opening 230 of the balloon 30 and bonding thereto using a well-known method, such as for example an ultraviolet-curable adhesive. Alternatively, the balloon 30 may be attached to the shaft 20 with the use of a solvent or by gluing. Other suitable methods of attachment are also contemplated. Specifically, the inner and the outer balloon layers can be bonded to the shaft simultaneously or individually (inner balloon layer and then the outer balloon layer).

Referring back to FIG. 2, the distal end 180 of the balloon 30 may have a standard tapered or domed configuration with a flexible tip 240. Alternatively, the distal end 180 of the balloon 30 may be formed so that it is generally truncate in shape, having a substantially flat end, rather than comprising standard configurations discussed above.

The balloon 30 is configured to be inflated to a predetermined or specific “inflated balloon diameter” or “outer balloon diameter.” The terms “inflated balloon diameter” or “outer balloon diameter” of the balloon 30 refer to the diameter of the outer most layer of the outer balloon layer and are specific or predetermined for a given balloon. Preferably, the inflated balloon diameter can fall within a range from about 2 millimeters to about 30 millimeters depending on the application of the balloon catheter and/or the medical procedure. More preferably, the inflated balloon diameter can fall within a range from about 3 millimeters to about 14 millimeters.

Also, although the above described balloon 30 may be configured to be inflated to a single predetermined or specific balloon diameter, due to variations in pressure, materials, environmental and other factors, the inflated balloon diameter may be slightly larger or slightly smaller than the single predetermined or specific diameter of the balloon 30. For example, for a balloon configured to have the single predetermined or specific inflated balloon diameter of 10 millimeters, the balloon is configured to be inflated to an inflated balloon diameter in the range from about 9.8 millimeters to about 10.2 millimeters.

Moreover, although, in the embodiments of the device described above, the balloon 30 can have a single predetermined inflated balloon diameter, the balloon 30 can be configured to be inflated to a plurality of predetermined or specific balloon diameters, each inflated balloon diameter being the result the pressure or the amount of inflation fluid delivered to the balloon 30.

The length of the balloon body 140 can be in a range of from about 2 centimeters to about 25 centimeters. Preferably, the length of the balloon body 140 can be made from about 2 centimeters to 14 centimeters.

The balloon will preferably have a burst pressure of at least 12 ATM; and more preferably at least 20 ATM; and most preferably as high as 30 ATM.

Referring to FIG. 3, which is a cross-sectional view though E-E of the balloon catheter shown in FIG. 2, the balloon body 140 of the balloon catheter 10 includes an inner balloon layer 190 and an outer balloon layer 200. The inner and the outer balloon layers 190, 200 are preferably made from a non-porous non-compliant balloon material, as described above with reference to the material that may be used to form the balloon 30.

The thickness of the inner balloon layer 190 may be in a range of from about 0.014 millimeters to about 0.060 millimeters, and preferably in a range of from about 0.020 millimeters to about 0.045 millimeters. The thickness of the outer balloon layer 200 may be in a range of from 0.008 millimeters to about 0.047 millimeters, and preferably in a range of from about 0.012 millimeters to about 0.035 millimeters.

The combined thickness of the inner and the outer balloon layers 190, 200 may be in a range of from about 0.032 millimeters to about 0.08 millimeters (not taking into account the thickness of the middle layer 210 disposed between the inner and the outer balloon layers 190, 200, as discussed below). Preferably, the combined thickness of the inner and the outer balloon layers 190, 200 may be in a range of from about 0.032 millimeters to about 0.07 millimeters. More preferably, the combined thickness of the inner and the outer balloon layers 190, 200 may be in a range of from about 0.032 millimeters to about 0.06 millimeters. Most preferably, the combined thickness of the inner and the outer balloon layers 190, 200 may be in a range of from about 0.032 millimeters to about 0.05 millimeters.

Also, the balloon 30 includes at least one additional middle layer 210, which is configured to be substantially free from adhesion to at least one of the inner balloon layer and the outer balloon layer and can comprise at least one layer of fluoropolymer, such as ePTFE, or other material, such as polyethylene disposed between the outer surface of the inner balloon layer 190 and the inner surface of the outer balloon layer 200 of the balloon body 140, as shown in FIGS. 2 and 3. Specifically, the middle layer 210 may be disposed between the inner and the outer balloon layers 190, 200 by positioning a piece of material, such as ePTFE over the inner balloon layer 190. Alternatively, at least one layer of the ePTFE 210 can be anchored into the bond area or left free floating. The ePTFE layer(s) 210 is not adhered to the inner or the outer balloon layers 190, 200 and allows independent expansion of each layer. The inner balloon layer 190 will expand the middle layer 210 during inflation.

Preferably, the ePTFE layer(s) is disposed over the entire outer surface of the inner balloon layer 190 and may be integral with the two balloon layers of the balloon body and connects the inner and the outer balloon layers of the balloon body, as illustrated in FIG. 3.

The thickness of the middle layer 210 may vary depending on the desired overall thickness of the balloon 30, the thickness of the two balloon layers 190, 200 that form the balloon body 140, and the application for which the balloon catheter is intended. Additionally, the thickness of the middle layer may further depend on the blow molding method selected to form the balloon. The desired thickness may be anywhere in the range from about 0.010 millimeters to about 0.070 millimeters.

The combined thickness of the inner balloon layer 190 and the outer balloon layer 200 will depend on the balloon size and application of the balloon catheter. Nonetheless, it is preferred that the inner balloon layer 190 is thicker than the outer balloon layer 200. In certain instances, it may be preferred for the outer balloon layer 200 to be thicker than the inner balloon layer 190. The inner and the outer balloon layers 190, 200 may also have the same thickness.

The number of ePTFE layers in the middle layer may also vary. For example, a balloon may include 1, 2, 3 or more ePTFE layers disposed between the inner and the outer balloon layers 190 and 200. In certain instances, the number of ePTFE layers can range from 1 to 5. In any event, the balloon will include at least one ePTFE layer disposed between the inner and the outer balloon layers.

By including the ePTFE layer 210 between the inner balloon layer 190 and the outer balloon layer 200, the balloon catheter 10 can be used for high pressure applications. The ePTFE layer 210 can advantageously allow the two balloon layers 190, 200 of the balloon body 140 to expand independently of each other during inflation of the balloon 30, while maintaining contact with each other. Because the layers 190, 200 are in contact with each other during inflation but moving independently, as discussed previously, the overall burst will be significantly higher than a single layer balloon having dimensions (e.g., thickness) equivalent to the dimensions of the two balloon layers of the balloon body combined. It is believed that this is because the stress on the inner diameter of the inner balloon layer is now distributed over another balloon layer (i.e., there is a larger surface area to distribute the stress). In addition, the ePTFE layer 210 can function to resist circular tearing of the balloon under burst pressure.

The balloon 30 also includes a balloon chamber 150 within the inner balloon layer 190 of the balloon body 140. The balloon chamber 150 is in communication with the lumen 100 of the shaft 20 for inflating and deflating the balloon 30.

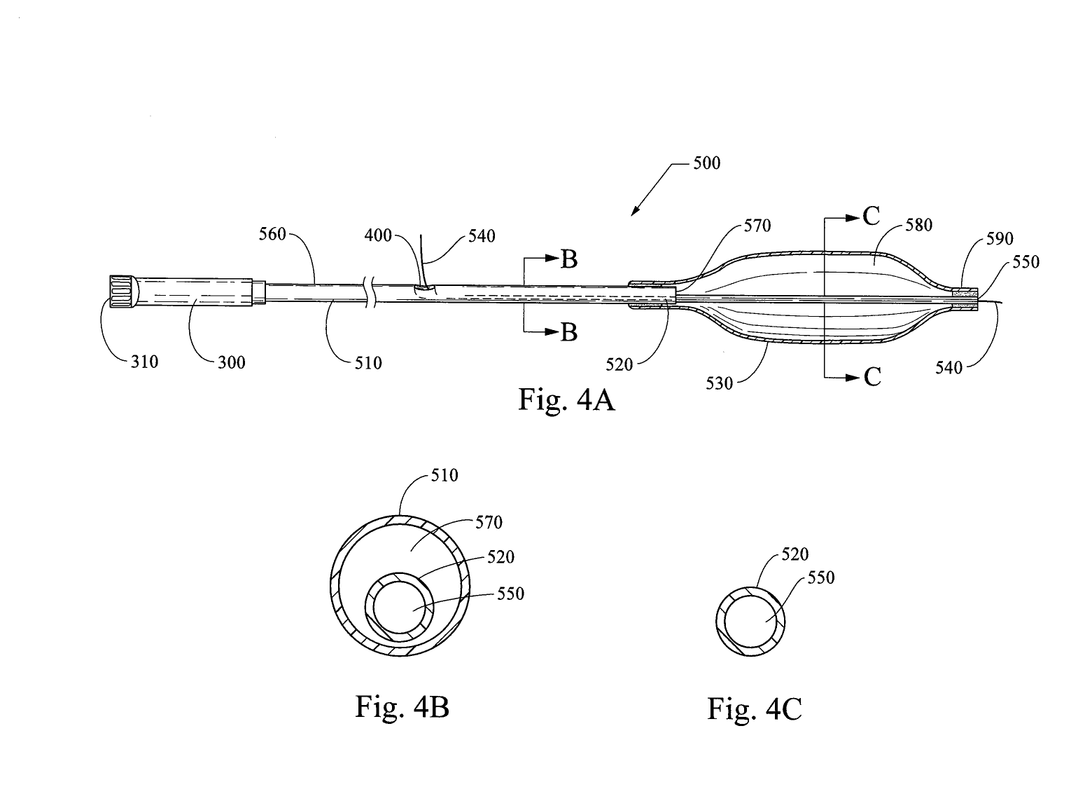

In one alternative embodiment illustrated in FIGS. 4A-C, the shaft 510 can have a coaxial configuration, where wire-guided dilation balloon catheter 500 includes an inner shaft 520 coaxially disposed within the main shaft 510 to which the balloon portion 530 is attached. Cross-sectional views though B-B and C-C of the balloon catheter of FIG. 4A are shown in FIGS. 4B and 4C, respectively. The inner shaft 520 serves as the conduit for the wire guide 540, which in one embodiment, is a standard 0.035″ wire guide that is loaded into, and is extendable from the inner shaft lumen 550. In the illustrative embodiment, both the inner and main shafts 520, 510 can be made of poly-ether ether ketone (PEEK). In other embodiments, a metal hypotube may be employed for all or at least the proximal portion 560 of the shaft 510. The inner and outer shafts 520, 510 are sized to allow the flow of inflation fluid within the annular space 570 between the two shafts 510, 520 and into the balloon chamber 580 of the balloon 530 to expand the balloon 530.

The inner shaft 520 can terminate within the distal end 590 of the balloon 530 or a few millimeters distally thereof. The wire guide 540 is typically utilized for adding stiffness or pushability to the balloon catheter 500, or it may be introduced separately into the patient and then used to guide the balloon catheter into the patient. The inner shaft 520 alone may provide sufficient stiffness and pushability for some applications. If desired, a wire guide 540 may at some point be replaced with a different wire guide having characteristics more desirable for a particular procedure. In the illustrative embodiment, the inner shaft 520 comprises a port 400 through a side wall thereof in communication with the wireguide lumen 550, the port being located proximal of the balloon 530 and a substantial distance from the proximal end 560 of the shaft 510. A standard hub 300 provides a port 310 for the infusion of a balloon inflation fluid, such as water or saline.

Alternatively, the outer and inner shafts may be fixed relative to one another longitudinally by a standard hub, which provides access for the wire guide, and a port for the infusion of a balloon inflation fluid, as described above in connection with a dual lumen shaft.

Various methods may be utilized to form the balloon of the balloon catheter described herein. Specifically, first, the inner and the outer balloon layers can be made according to the following process. The balloon material is first extruded into a suitable shape by a well-known means, such as blow molding, whereby a length of Nylon tubing, sufficient in length to form the final desired length of the balloon layer, is placed and clamped within a mold conforming to the final shape of the fully distended balloon layer. The extruded balloon material is then placed into a forming mold to blow mold the balloon layers. Hot air is passed through the tubing, causing the tubing to expand against the contours of the mold. The tubing and molding process parameters necessary to achieve the desired balloon layer are determined by the required burst strength and recommended pressure of the balloon layer, the material used, and the size of the balloon layer. One source of the balloon portion of the illustrative embodiment is Advanced Polymers, Inc. (Salem, N.H.).

FIG. 6 depicts a standard single lumen balloon tubing.

After each of the inner and the outer balloon layers are formed individually as described above, the middle layer comprising, for example, ePTFE layer may be incorporated in the balloon portion of the balloon catheter. There are a few possible methods of incorporating the ePTFE. In one exemplary method, the inner balloon layer may first be bonded to the shaft and then folded. Then an extruded ePTFE tube may be placed over the inner balloon layer. Next the outer balloon layer may be placed over the ePTFE tubing and then bonded to the shaft. The outer balloon layer may then be folded. The ePTFE tube would be inflated during actual use of the device. Alternatively, the inner balloon layer may first be bonded to the shaft and then folded. Then an extruded ePTFE tube may be placed over the inner balloon layer. The inner balloon layer is then used to expand the ePTFE tubing. Next the outer balloon layer is placed over the inner balloon layer and the ePTFE layer, and then bonded to the shaft. The entire assembly may be then folded simultaneously. In yet another method, the ePTFE tube may be placed over the inner balloon layer. The balloon and ePTFE tube may then be placed back into the balloon forming mold and pressurized to expand the ePTFE tube. This ensures that the ePTFE tube is formed into the exact shape of the balloon. The inner balloon layer is then bonded to the shaft. The outer balloon layer may then be placed over the inner balloon layer and the ePTFE layer, and then bonded to the shaft. The entire assembly may then be folded simultaneously.

In an exemplary method of using the balloon catheter device of the present invention, to dilate a stricture, a small incision is made in the patient to facilitate the insertion of a long, thin introducer sheath. A guide catheter is then passed through the sheath and into the narrowed artery. The physician may monitor the insertion of the guide catheter under fluoroscopy. An injection through the guide catheter of contrast dye/medium allows the physician visualization of the peripheral arteries.

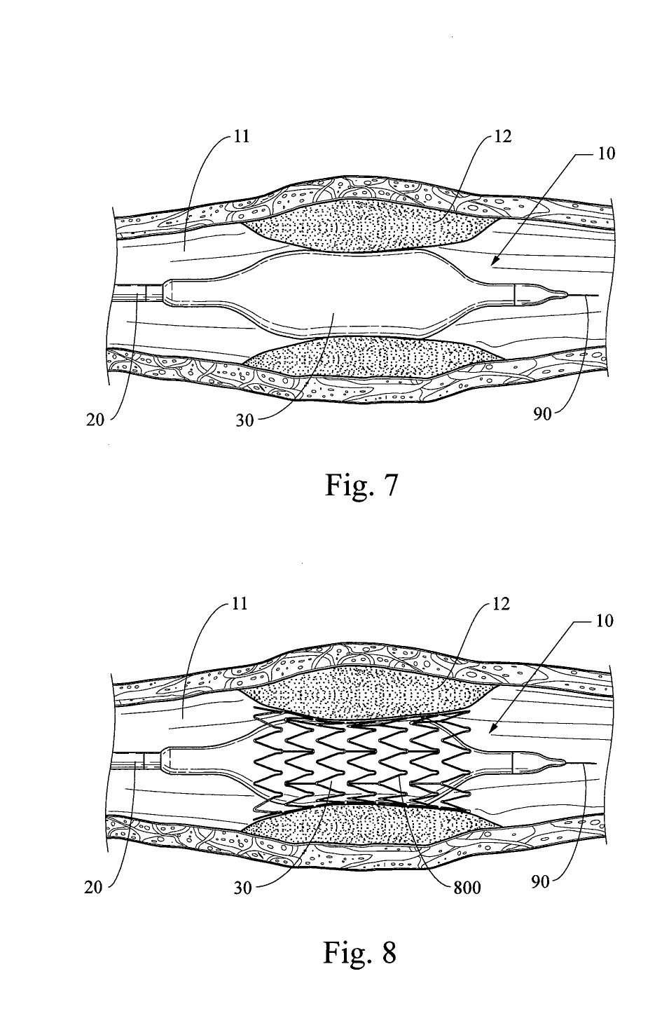

Once the guide catheter is engaged in the ostium of the artery where the lesion/vessel stricture is located, a wire guide is threaded through the guide catheter. The wire guide is then advanced under fluoroscopy beyond the lesion to a distal location within the artery. With the wire guide in place, the dilation balloon catheter of the present invention is inserted over the wire guide and advanced to the lesion site, as illustrated in FIGS. 7 and 8.

Referring to FIG. 7, once the balloon catheter 10 comprising a shaft 20 and a balloon 30, and optionally a wireguide 90, has been properly positioned in the bodily lumen 11, the balloon 30 is dilated within the artery at the lesion/stricture site 12, causing a compression of the arterial plaque against the inner lining of the arterial wall. Subsequent balloon dilation may be used if the physician decides to increase the atmospheres of pressure or duration of time that the balloon is applied to the lesion.

Referring to FIG. 8, in addition or alternatively, the exemplary device of this invention may be used to expand and deploy a stent 800. Specifically, upon examination of the pre and post PTA images, the physician may decide to follow the PTA procedure with the implantation of a stent 800 at the site of the lesion. A stent 800 may be provided, which can then be compressed about the balloon 30 when the balloon is in an uninflated state. Once in position, the balloon can be expanded to expand and deploy the stent.

The physician may also consider using an adjunctive imaging device such as intravascular ultrasound (IVUS). This provides the physician with a cross-sectional and longitudinal image of the vessel and morphology of the plaque. IVUS allows for measurement of the artery and the plaque burden, which assists the physician with accurate sizing of the stent to be used.

It will be appreciated that the devices described herein will be useful in catheters, particularly high-pressure vascular balloon catheters, other types of medical procedures and in various types of balloons, wherein they will provide structural strength to resist bursting under pressure, torsional and longitudinal directivity and kink resistance while maintaining the small diametric profile necessary for traversing small tortuous vascular channels.

All patents, patent applications, provisional applications, and publications referred to or cited herein are incorporated by reference in their entirety, including all figures and tables, to the extent they are not inconsistent with the explicit teachings of this specification.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

1. A dilation balloon catheter comprising:
an elongate shaft extending between a proximal end and a distal end, the proximal end being adapted for attachment to a source of inflation fluid, and a lumen extending through the shaft adapted for the passage of the inflation fluid;
a balloon disposed on the distal end of the shaft and having a balloon body extending between a proximal end and a distal end of the balloon, the balloon body comprising:
an inner balloon layer;
an outer balloon layer,
a middle layer disposed between the inner balloon layer and the outer balloon layer and configured to be substantially free from adhesion to at least one of the inner balloon layer and the outer balloon layer, and
a balloon chamber within the inner balloon layer, the balloon chamber being in a communication with the lumen of the shaft for inflating and deflating the balloon.

2. The dilation balloon catheter of claim 1, wherein the middle layer comprises a proximal end and a distal end, the middle layer being connected to the inner balloon layer and the outer balloon layer at the proximal and distal ends.

3. The dilation balloon catheter of claim 1, wherein the inner balloon layer and the outer balloon layer are formed from a substantially non-compliant, non-porous elastomeric material.

4. The balloon catheter of claim 3, wherein the substantially non-compliant and non-porous elastomeric material is selected from the group consisting of Nylon (Nylon 12), polyether block amide (PEBAX), PEBAX 4033, PEBAX 5533, PEBAX 6333, and poly(ethylene terephthalate) (PET).

5. The dilation balloon catheter of claim 1, wherein the balloon has a predetermined inflated diameter in the range of from about 2 millimeters to about 30 millimeters.

6. The dilation balloon catheter of claim 5, wherein the inner balloon layer is configured to have the inflated diameter smaller than the inflated diameter of the outer balloon layer.

7. The dilation balloon catheter of claim 1, wherein the inner balloon layer has thickness from about 0.014 millimeters to about 0.060 millimeters.

8. The dilation balloon catheter of claim 1, wherein the outer balloon layer has thickness from about 0.008 millimeters to about 0.047 millimeters.

9. The dilation balloon catheter of claim 1, wherein the middle layer has thickness from about 0.010 millimeters to about 0.070 millimeters.

10. The dilation balloon catheter of claim 1, wherein the proximal and the distal ends of the balloon body are each tapered.

11. The dilation balloon catheter of claim 1, wherein the inner and the outer balloon layers comprise different materials.

12. The dilation balloon catheter of claim 1, wherein the inner and the outer balloon layers comprise different thicknesses.

13. The dilation balloon catheter of claim 1, wherein the shaft further comprises a wireguide lumen extending through at least a portion thereof.

14. The dilation balloon catheter of claim 1, wherein the middle layer comprises a fluoropolymer.

15. The dilation balloon catheter of claim 14, wherein the fluoropolymer is expanded polytetrafluorethylene.

16. A method for dilating a vessel stricture comprising:
providing the dilation balloon catheter of claim 1;
positioning the balloon within or near the vessel stricture; and
inflating the balloon to dilate or widen the vessel stricture.

17. The method of claim 16, further comprising the steps of:
providing a stent;
compressing the stent about the balloon when the balloon is in an uninflated state; and
expanding the balloon to expand and deploy the stent.

18. A dilation balloon catheter comprising:
an elongate shaft extending between a proximal end and a distal end, the proximal end being adapted for attachment to a source of inflation fluid, and a lumen extending through the shaft adapted for the passage of the inflation fluid;
a balloon disposed on the distal end of the shaft and having a balloon body extending between a proximal end and a distal end of the balloon, the balloon body comprising:
an inner balloon layer;
an outer balloon layer,
at least one layer of fluoropolymer disposed between the inner balloon layer and the outer balloon layer, and
a balloon chamber within the inner balloon layer, the balloon chamber being in a communication with the lumen of the shaft for inflating and deflating the balloon.

19. The dilation balloon catheter of claim 18, wherein the inner balloon layer and the outer balloon layer are formed from a substantially non-compliant, non-porous elastomeric material.

20. The dilation balloon catheter of claim 18, wherein the at least one layer of fluoropolymer comprises at least one layer of expanded polytetrafluorethylene.
